(19) United States
(12) Patent Application Publication
(10) Pub. No.: US 2006/0271077 A1
(43) Pub. Date: Nov. 30, 2006

(54) Combination scalpel blade

(76) Inventor: Robert E. Graser, San Antonio, TX (US)

Correspondence Address: KAMMER BROWNING PLLC, 7700 BROADWAY, SUITE 202, SAN ANTONIO, TX, 78209, US

(21) Appl. No.: 11/141,600

(22) Filed: May 31, 2005

(51) Int. Cl. A61B 17/32

(52) U.S. Cl. 606/167

(57) ABSTRACT

A surgical scalpel having multiple cutting segments designed especially for podiatric surgical procedures is disclosed. More particularly, the scalpel blade has a handle attachment section and a number of cutting segments. The blade includes a single-edged longitudinal cutting segment extending from the handle attachment section, a single-edged curved cutting segment contiguous with the longitudinal cutting segment, and a double-edged end tip cutting segment contiguous with the curved cutting segment. The double-edged end tip cutting segment may be designed in varying degrees of curvature, including semi-circular and semi-elliptical. Multiple cutting edges are contained on one scalpel blade for optimum flexibility, efficiency, and economy.

### BACKGROUND OF THE INVENTION

1. Field of the Invention

The present invention relates generally to surgical scalpel blades. The present invention relates more specifically to a scalpel blade designed especially for podiatric surgical procedures.

Applicant's invention improves upon the state of the art through an innovative combination of multiple cutting edges on one scalpel blade for optimum flexibility, efficiency, and economy.

2. Description of the Related Art

Scalpel blades are utilized in a wide variety of medical procedures, particularly for soft tissue surgery. Traditionally, the surgical scalpel was designed as a single unit having a long handle joined to a cutting blade which extended from the handle. The blade was shaped for a specific type of surgical procedure and the instrument was subsequently re-sterilized and re-used. Modern surgical scalpels are frequently designed to use disposable blades and comprise a handle having a blade-fitting end with a protrusion for detachable engagement with a hole in the disposable blade. The disposable blades come in various types and shapes and are selected according to the specific surgical procedure to be performed.

One problem with the disposable/replaceable blade scalpel is that the work of replacing the blade with another requires meticulous precision due to the small blade size. The disposable blades are sharp, curved, small and difficult to detach and replace. Moreover, the used blade is often slippery from body fluids deposited on the blade during the surgical procedure.

In the prior art, there are various types of scalpels which are designed for surgery on a specific area of the body and/or a specific procedure on that area. For example, there are multiple types of scalpels designed for ophthalmic surgery. U.S. Pat. No. 4,516,575 for Surgical Scalpel issued May 14, 1985 to Gerhard et al., discloses a scalpel having a blade that can be adjustably extended with an indicator for showing the length of the blade that is exposed beyond the foot of the scalpel. The indicator can be calibrated to compensate for variations in blade length. U.S. Pat. No. 5,224,950 for Color Calibrated Multi-function Scalpel Blade for Intraocular and Other Surgery and Associated Methods of Use issued Jul. 6, 1993 to Prywes, teaches a scalpel having a blade member with a plurality of juxtaposed cutting sections arranged successively along the blade member. The various cutting sections are visually delineated by coloration. U.S. Pat. No. 5,078,724 for Scalpel issued Jan. 7, 1992 to Takase, describes a scalpel having a disposable blade and a handle for retaining the blade. The disposable blade is provided on its rear end portion with at least one tongue piece which can be held with a tool such as a needle holder or forceps.

In general, multiple blade types are required to perform a single podiatric procedure. For example, various steps in the surgical procedure such as initial incision of the skin and connective tissue, tissue or tumor excision, contouring of a protuberance, curettage of a tissue cavity, and debridement of foreign or infected matter may each require different types of scalpel blades. It would be desirable if a single disposable blade possessed a variety of cutting edges such that it would not be necessary to attach and detach multiple blades during the surgical procedure. With such an instrument, the surgeon would be able to manipulate a single scalpel to perform various cutting tasks according to the application, thus eliminating the need to replace the blade with another or to switch back and forth between scalpels in the midst of a surgical operation.

### SUMMARY OF THE INVENTION

The surgical scalpel of the present invention is designed to provide an innovative combination of multiple cutting edges on one scalpel blade for optimum flexibility, efficiency, and economy. The scalpel blade is specifically designed for the types of cutting required for podiatric surgical procedures. More particularly, the scalpel blade has a handle attachment section and a plurality of cutting segments. The blade comprises a series of cutting edges: a single-edged longitudinal cutting segment extending from the handle attachment section; a single-edged curved cutting segment contiguous with and extending from the longitudinal cutting segment; and a double-edged end tip cutting segment contiguous with the curved cutting segment.

In a variety of podiatric surgical operations, it is frequently necessary to use a combination of scalpel blades in order to accomplish multiple types of cutting procedures. Three prior art scalpels are shown in FIGS. 3A, 3B, and 3C. FIG. 3A illustrates a #10 scalpel blade having a broad cutting edge with an extended longitudinal segment, FIG. 3B illustrates a #15 scalpel blade having a shorter cutting edge with a curved cutting segment, and finally, FIG. 3C illustrates a Beaver® blade having a pointed end tip cutting segment. For example, in a relatively simple procedure such as the removal of a plantar wart, the podiatrist may decide to perform surgical excision of the wart because this treatment has a high success rate with a relatively low rate of recurrence of the condition. The surgical removal or curettage of the wart, involves the scraping or cutting of the wart tissue with a scalpel. In this procedure, the podiatrist may use a straight sharp blade for the initial incision, a pointed or curved blade for the excision of the wart itself, and a curved sharp blade to scrape away any remnants of the wart tissue. It would therefore be desirable to have a single scalpel having a blade containing a combination of cutting surfaces such that the podiatrist could move smoothly from one step of the surgical procedure to the next without the delay and inefficiency involved in switching between scalpels or changing scalpel blades.

Similarly, the removal of a neuroma requires the podiatrist to perform multiple surgical steps for the removal of the damaged nerve. The podiatrist most often will incise the top of the patient's foot, locate the neuroma between the metatarsals behind the toes, and then cut the nerve and remove it. These steps require a blade for the initial incision, a blade for cutting the nerve, and a blade for removal of the nerve from the patient's foot. Once again, it would be helpful to have a single blade with multiple cutting edges to perform all of these tasks without having to utilize multiple scalpels or blades.

Even for more complicated procedures, such as surgery for severe chronic Achilles tendonitis or a bunionectomy, it would be useful to have a multi-purpose cutting blade to simplify the cutting procedures as much as possible. For example, in the surgery for Achilles tendonitis, the Achilles tendon is explored, partial tears are surgically debrided, and the remaining tissue is repaired. The thickened tissue is incised or excised, and any bony prominence is excised. Although this surgical procedure may require multiple instruments, it would still be beneficial to have a single scalpel that could be used to perform several of the necessary cutting tasks, including incision, excision, scraping, and nodular tissue removal.

In a bunionectomy, the bump of bone is removed from the side of the first metatarsal head. The podiatric surgeon then creates an osteotomy through the first metatarsal that permits shifting the bone and realigning the joint. Because the bone is cut and repositioned, the surgeon will often prefer to fixate the bone in place with an external device or pin across the bone cut. Alternatively, small screws are used to provide compression of the bone and to hold the bone in position. Although this procedure will require multiple instruments, various aspects of the surgery could be simplified by the use of a scalpel having multiple cutting edges designed for discrete purposes. For example, the slicing edge could be used for the initial incision of the skin and soft tissue, the double-edged tip cutting segment could be used for entry into the joint area, and the curved edge could be used for removal of excess tissue in the surgical area.

The present invention satisfies the above described need for a combination scalpel having a plurality of cutting edges which are designed for discrete surgical tasks such as incision, excision, curettage, debridement, and removal of a nodular mass.

### BRIEF DESCRIPTION OF THE DRAWINGS

FIG. 1 is a diagrammatic side view of the scalpel of the present invention having a semi-circular arc end tip cutting segment;

FIG. 2 is a diagrammatic side view of the scalpel of the present invention having a semi-elliptical arc end tip cutting segment;

FIG. 3A is a diagrammatic side view of a #10 scalpel of the prior art;

FIG. 3B is a diagrammatic side view of a #15 scalpel of the prior art; and

FIG. 3C is a diagrammatic side view of a Beaver® blade scalpel of the prior art.

### DETAILED DESCRIPTION OF THE PREFERRED EMBODIMENTS

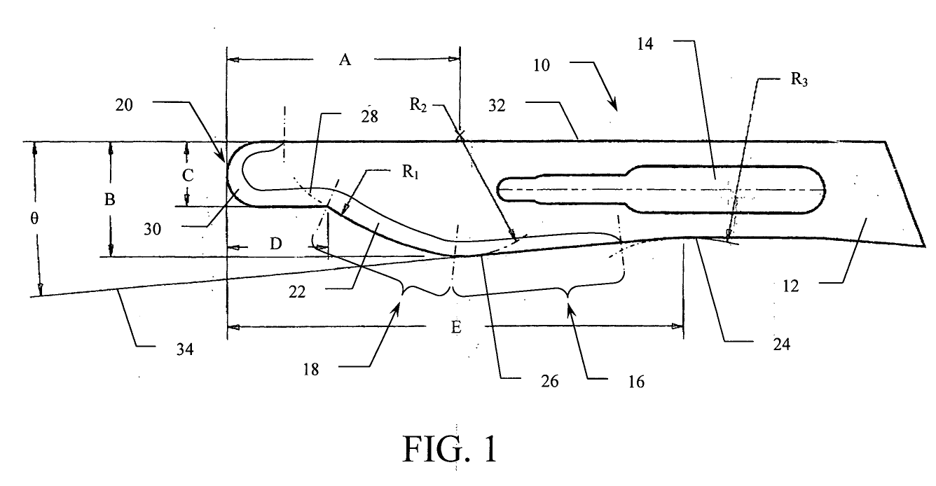

Reference is made first to FIG. 1 for a detailed description of the structure and function of a scalpel blade according to the present invention. FIG. 1 illustrates a surgical scalpel blade 10 that includes a blade base 12 with a standard handle attachment slot 14. The combination blade 10 further includes a longitudinal cutting segment 16, a curved cutting segment 18, and an end tip cutting segment 20 positioned along the sharpened edge 22 of the blade. The structure of the blade is such that the three cutting surfaces are contiguous, with the longitudinal cutting segment 16 positioned next to the curved cutting segment 18 which is adjacent to the end tip cutting segment 20.

The blade base 12 transitions into the longitudinal cutting segment 16 through a short circular arc transition segment 24 having a radius R₃ of approximately 0.5 inches in the preferred embodiment. The longitudinal cutting segment 16 comprises a straight section of cutting (sharpened) edge that is positioned at an angle θ with respect to a top edge 32 of the blade 10. This angle θ in the preferred embodiment is approximately 5.0°. The longitudinal cutting segment 16 transitions into the curved cutting segment 18 through a circular arc transition segment 26 having a radius R₂ in the range of 0.2-0.3 inches in the preferred embodiment. The curved cutting segment 18 itself comprises a cutting edge that is formed from a circular arc having a radius R₁ in the range of 0.7-0.8 inches in the preferred embodiment. The end tip cutting segment 20 as shown in FIG. 1 has a semi-circular arc cutting edge 30 having a diameter C in the preferred range of 0.10-0.15 inches.

Referring still to FIG. 1, the overall length E from the distal end of the end tip cutting segment 20 to the midpoint of circular arc transition segment 24 is approximately 0.98 inches in the preferred embodiment. The length A from the distal end of the end tip cutting segment 20 to the midpoint of circular arc transition segment 26 is approximately 0.50 inches in the preferred embodiment. The length D from the distal end of the end tip cutting segment 20 to the circular arc transition segment 28 is preferably approximately 0.22 inches. The width B of the overall blade 10 at its widest point is approximately 0.24 inches and the width C of end tip cutting segment 20 is approximately 0.13 inches in the preferred embodiment.

The longitudinal cutting segment 16 is typically used for tissue incision or excision. The curved cutting segment 18 is designed to facilitate excision of tissue such as scraping or curettage within an incised area or cavity. The end tip cutting segment 20 is designed for removal of nodular tissue or lesions.

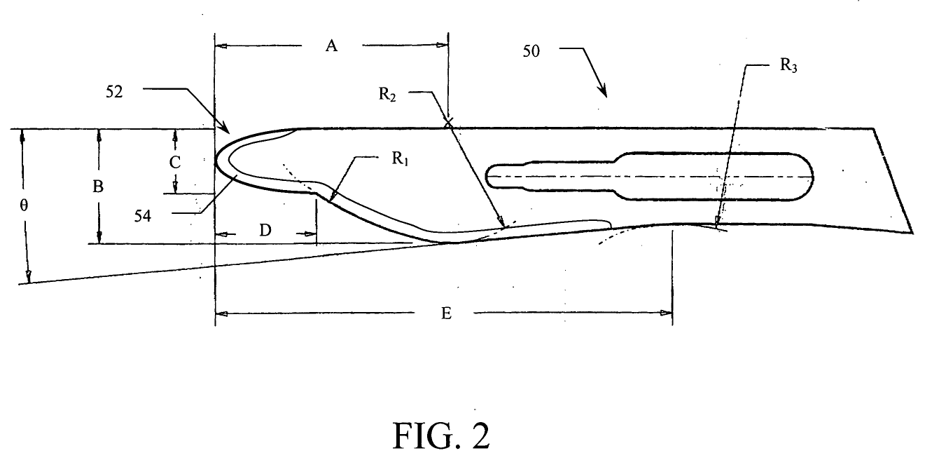

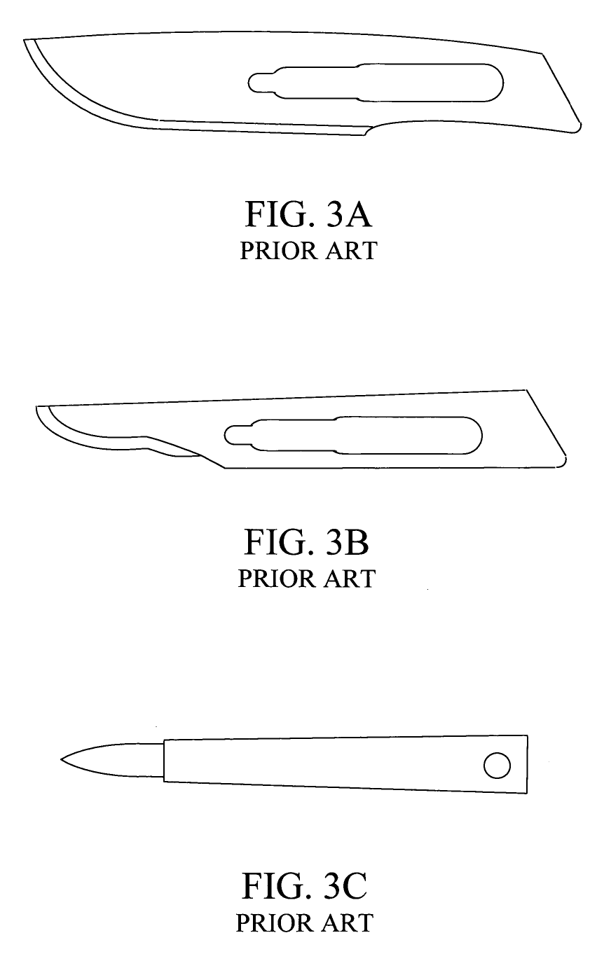

Alternatively, as shown in FIG. 2, another embodiment of the surgical scalpel blade 50 has a different end tip cutting segment 52. In this blade configuration, the end tip is shaped with a semi-elliptical arc cutting edge 54. The semi-elliptical arc cutting edge 54 of the cutting segment 52 has a major axis at least two times its minor axis in the preferred embodiment. As illustrated in FIG. 2, this surgical scalpel blade 50 possesses all of the characteristics and dimensions of the scalpel blade 10 disclosed in FIG. 1, with the exception that the end tip cutting segment 20 of FIG. 1 forms a semi-circular arc cutting edge 30, and the end tip cutting segment 52 of FIG. 2 forms a semi-elliptical arc cutting edge 54. This semi-elliptical shape is designed to facilitate the removal of smaller, more compact or punctate lesions or areas of tissue.

Although the present invention has been described in conjunction with preferred embodiments, it will be understood by those skilled in the art that alternative embodiments are possible without departing from the fundamental concept of the present invention. As indicated above, the choice of a specific cutting segment to be utilized in conjunction with the present invention is best determined by the specific application of the surgical device. Likewise, the selection of the combination of blade components of the present invention will be a matter of choice dependent on the particular podiatric procedure to be performed.

Moreover, the examples of scalpel blade cutting edges and combinations of those cutting edges described above are not intended to be limitations on the possible applications of a device constructed according to the present invention. Those skilled in the art will readily recognize many other applications that could benefit from the use of a device having the variable combination blade shapes of the present invention.

I claim:

1. A scalpel blade having a handle attachment section and a plurality of cutting segments, the blade comprising:
a single edged, longitudinal cutting segment extending from said handle attachment section;
a single edged, curved cutting segment extending from said longitudinal cutting segment; and
a double edged, end tip cutting segment extending from said curved cutting segment.

2. The scalpel blade of claim 1 wherein said longitudinal cutting segment transitions to said curved cutting segment through a circular arc transition segment.

3. The scalpel blade of claim 2 wherein said circular arc transition segment has a radius in the range from 0.2″-0.3″.

4. The scalpel blade of claim 1 wherein said curved cutting segment comprises a circular arc cutting edge.

5. The scalpel blade of claim 4 wherein said circular arc cutting edge has a radius in the range from 0.70″-0.80″.

6. The scalpel blade of claim 1 wherein said double edged end tip cutting segment comprises a semi-circular arc cutting edge.

7. The scalpel blade of claim 6 wherein said semi-circular arc cutting edge has a diameter in the range from 0.10″-0.15″.

8. The scalpel blade of claim 1 wherein said double edged end tip cutting segment comprises a semi-elliptical arc cutting edge.

9. The scalpel blade of claim 8 wherein said semi-elliptical arc cutting edge has a major axis at least two times its minor axis.
